(19) United States
(12) Patent Application Publication
(10) Pub. No.: US 2016/0097086 A1
(43) Pub. Date: Apr. 7, 2016

(54) Compositions and Methods for RT-PCR

(71) Applicant: Jun Euihum Lee, San Diego, CA (US)

(72) Inventor: Jun Euihum Lee, San Diego, CA (US)

(21) Appl. No.: 14/506,573

(22) Filed: Oct. 3, 2014

(51) Int. Cl. C12Q 1/68

(52) U.S. Cl. CPC C12Q 1/686; C12Q 1/6851

(57) ABSTRACT

The present invention relates to compositions and methods having propylene glycol and DNA polymerase for facilitating the rapid and efficient amplification of nucleic acid molecules and the detection and quantitation of RNA molecules, and for increasing the detection sensitivity and reliability through generation of secure cDNA molecules prior to gene-specific primer dependent amplification. The reagent mixture comprises a ready to use reagent solution, wherein the solution comprises: (a) propylene glycol in a concentration between about 20% and about 50%; (b) a viral reverse transcriptase; and (c) at least one DNA polymerases, in a buffer suitable for use in a reverse transcription reaction, wherein the buffer comprises a co-factor metal ion and nucleoside triphosphates.

### FIELD OF THE INVENTION

The present invention provides compositions and methods for preparing RT-PCR, and more specifically, compositions having propylene glycol and deoxyribonucleic acids (DNA) polymerase for facilitating the rapid and efficient amplification of nucleic acid molecules and the detection and quantitation of ribonucleic acid (RNA) molecules, and for increasing the detection sensitivity and reliability through generation of secure complementary deoxyribonucleic acid (cDNA) molecules prior to gene-specific primer dependent amplification.

### BACKGROUND OF THE INVENTION

Within a given cell, tissue or organism, there exist many messenger ribonucleic acid (mRNA) species, each encoding a separate and specific protein. The identity and levels of specific mRNAs present in a particular sample provides clues to the biology of the particular tissue or sample being studied. Therefore, the detection, analysis, transcription, and amplification of RNAs are among the most important procedures in modern molecular biology.

A common approach to the study of gene expression is the production of complementary DNA (cDNA). In this technique, the mRNA molecules from an organism are isolated from an extract of the cells or tissues of the organism. From these purified mRNA molecules, cDNA copies may be made using the enzyme reverse transcriptase (RT), which results in the production of single-stranded cDNA molecules.

The term “reverse transcriptase” describes a class of polymerases characterized as RNA-dependent DNA polymerases. All known reverse transcriptases require a primer to synthesize a DNA transcript from an RNA template. Historically, reverse transcriptase has been used primarily to transcribe mRNA into cDNA which can then be cloned into a vector for further manipulation.

Avian myoblastosis virus (AMV) reverse transcriptase was the first widely used RNA-dependent DNA polymerase (Verma, Biochim. Biophys. Acta 473:1 (1977)). The enzyme has 5′-3′ RNA-directed DNA polymerase activity, 5′-3′ DNA-directed DNA polymerase activity, and RNase H activity. RNase H is a processive 5′ and 3′ ribonuclease specific for the RNA strand for RNA-DNA hybrids (Perbal, A Practical Guide to Molecular Cloning, New York: Wiley & Sons (1984)). Errors in transcription cannot be corrected by reverse transcriptase because known viral reverse transcriptases lack the 3°.fwdarw.5′ exonuclease activity necessary for proofreading (Saunders and Saunders, Microbial Genetics Applied to Biotechnology, London: Croom Helm (1987)). A detailed study of the activity of AMV reverse transcriptase and its associated RNase H activity has been presented by Berger, et. al., Biochemistry 22:2365-2372 (1983).

Another reverse transcriptase which is used extensively in molecular biology is reverse transcriptase originating from Moloney murine leukemia virus (M-MLV). See, e.g., Gerard, G. R., DNA 5:271-279 (1986) and Kotewicz, M. L., et. al., Gene 35:249-258 (1985). M-MLV reverse transcriptase substantially lacking in RNase H activity has also been described. See, e.g., U.S. Pat. No. 5,244,797.

Reverse transcriptases have been extensively used in reverse transcribing RNA prior to PCR amplification. This method, often referred to as RNA-PCR or RT-PCR, is widely used for detection and quantitation of RNA.

To attempt to address the technical problems often associated with RT-PCR, a number of protocols have been developed taking into account the three basic steps of the procedure: (a) the denaturation of RNA and the hybridization of reverse primer; (b) the synthesis of cDNA; and (c) PCR amplification. In the so-called “uncoupled” RT-PCR procedure (e.g., two-step RT-PCR), reverse transcription is performed as an independent step using the optimal buffer condition for reverse transcriptase activity. Following cDNA synthesis, the reaction is diluted to decrease MgCl₂ and deoxyribonucleoside triphosphate (dNTP) concentrations to conditions optimal for Taq DNA Polymerase activity, and. PCR is carried out according to standard conditions (see U.S. Pat. Nos. 4,683,195 and 4,683,202). By contrast, “coupled” RT-PCR methods use a common or compromised buffer for reverse transcriptase and Taq DNA Polymerase activities. In one version, the annealing of reverse primer is a separate step preceding the addition of enzymes, which are then added to the single reaction vessel. In another version, the reverse transcriptase activity is a component of the thermostable Tth DNA polymerase. Annealing and cDNA synthesis are performed in the presence of Mn⁺⁺, then PCR is carried out in the presence of Mg⁺⁺ after the removal of Mn⁺⁺ by a chelating agent. Finally, the “continuous” method (e.g., one-step RT-PCR) integrates the three RT-PCR steps into a single continuous reaction that avoids the opening of the reaction tube for component or enzyme addition. Continuous RT-PCR has been described as a single enzyme system using the reverse transcriptase activity of thermostable Taq DNA Polymerase and Tth polymerase and as a two-enzyme system using AMV-RT and Taq DNA Polymerase wherein the initial 65 degree. RNA denaturation step was omitted.

Attempts to streamline the process of RT-PCR have not been easy, and several reports have documented an interference between reverse transcriptase and thermostable DNA polymerase Taq when used in combination in a single tube RT-PCR resulting in low sensitivity or lack of results. For example, there has been at least one report of a general inhibition of Taq DNA polymerase when mixed with reverse transcriptases in one-step/one tube RT-PCR mixtures (Sellner, L. N., et. al., Nucl. Acids Res. 20(7):1487-1490 (1992)). This same report indicated that the inhibition was not limited to one type of RT: both AMV-RT and M-MLV-RT inhibited Taq DNA polymerase and limited the sensitivity of RT-PCR. Under the reaction conditions used in the Sellner, et. al.

Other reports describe attempts to develop conditions for one-step RT-PCR reactions. For example, the use of AMV-RT for one-step RT-PCR in a buffer comprising 10 mM Tris-HCl, (pH 8.3), 50 mM KCl, 1.5 mM MgCl.sub.2, and 0.01% gelatin has been reported (Aatsinki, J. T., et al., BioTechniques 16(2):282-288 (1994)), while another report demonstrated one-step RT-PCR using a composition comprising AMV-RT and Taq DNA polymerase in a buffer consisting of 10 mM Tris-HCl, pH 8.3, 50 mM KCl, 0.01% gelatin and 1.5 mM MgCl₂ (Mallet, F., et. al., Biotechniques 18(4):678-687 (1995)). Under the reaction conditions used in the latter report, substitution of M-MLV-RT (RNase H.sup.+ or RNase H.sup.− forms) for AMV-RT showed the same activity in the continuous RT-PCR reaction.

RT-PCR system exploits the high sensitivity and specificity of the PCR process and is widely used for detection and quantification of RNA. Recently, the ability to measure the kinetics of a PCR reaction by on-line detection in combination with these RT-PCR techniques has enabled accurate and precise measurement of RNA sequences with high sensitivity. This has become possible by detecting the RT-PCR product through fluorescence monitoring and measurement of PCR product during the amplification process by fluorescent dual-labeled hybridization probe technologies, such as the “TaqMan” 5′ fluorogenic nuclease assay described by Holland et al. (Proc. Natl. Acad. Sci. U.S.A. 88, 7276 (1991)), and Gibson, et. al. (Genome Res. 6, 99 (1996) or “Molecular Beacons” (Tyagi, S. and Kramer, F. R. Nature Biotechnology 14, 303 (1996)) has described use of dual-labeled hairpin primers. One of the more widely used methods is the addition of double-strand DNA-specific fluorescent dyes to the reaction such as SYBR Green I (Wittwer, et. al., Biotechniques 22,130 (1997). These improvements in the PCR method have enabled simultaneous amplification and homogeneous detection of the amplified nucleic acid without purification of PCR product or separation by gel electrophoresis. This combined approach decreases sample handling, saves time, and greatly reduces the risk of product contamination for subsequent reactions, as there is no need to remove the samples from their closed containers for further analysis. The concept of combining amplification with product analysis has become known as “real time” PCR, also referred to as quantitative PCR, or qPCR. The general principles for template quantification by real-time PCR were first disclosed by Higuchi R, G Dollinger, P S Walsh and R. Griffith. Use of real time PCR methods provides a significant improvement towards this goal. However, real-time PCR quantification of mRNA is still bounded by limitations of the process of reverse transcription.

1-step RT-PCR provides several advantages over uncoupled RT-PCR. 1-step RT-PCR requires less handling of the reaction mixture reagents and nucleic acid products than uncoupled RT-PCR (e.g., opening of the reaction tube for component or enzyme addition in between the two reaction steps), and is therefore less labor intensive, reducing the required number of work hours. 1-step RT-PCR also requires less sample, and reduces the risk of contamination (Sellner and Turbett, 1998). The sensitivity and specificity of 1-step RT-PCR has proven well suited for studying expression levels of one to several genes in a given sample or the detection of pathogen RNA.

In contrast, use of non-specific primer in the “uncoupled” RT-PCR procedure provides opportunity to capture all RNA sequences in a sample into first-strand cDNA, thus enabling the profiling and quantitative measurement of many different sequences in a sample, each by a separate PCR. The ability to increase the total amount of cDNA produced, and more particularly to produce cDNA that truly represents the mRNA population of the sample would provide a significant advance in study of gene expression. Specifically, such advances would greatly improve the probability of identifying genes which are responsible for disease in various tissues.

### SUMMARY OF THE INVENTION

The present invention provides a method and kit for amplification of nucleic acid molecules by RT-PCR. Specifically, the invention provides compositions and methods for the amplification of nucleic acid molecules in a simplified PCR amplification procedure using combinations of reverse transcriptase and thermostable DNA polymerase enzymes in conjunction mixture of primers with gene-specific primers. Compositions are also provided comprising mixtures of reagents, including reverse transcriptases, thermostable DNA polymerases, buffers, cofactors and other components, suitable for immediate use in conversion of RNA into RT-PCR molecules without dilution or addition of further components. The invention also is useful in the rapid and non-bias production of cDNAs and subsequent amplification of gene specific molecules without dilution or addition of further components which may be used for a variety of research, medical, diagnostic, forensic and agricultural applications.

The invention thus facilitates the rapid and efficient amplification of nucleic acid molecules and the detection and quantitation of RNA molecules.

The present invention is directed to compositions comprising mixtures of reagents, including reverse transcriptases, DNA polymerases, buffers, cofactors and other components, suitable for immediate use in conversion of RNA into cDNA and PCR amplification without dilution or addition of further components. These compositions are useful, alone or in the form of kits, for nucleic acid amplification or for any procedure utilizing RT-PCR in a variety of research, medical, diagnostic, forensic and agricultural applications.

The invention also provides improved methods of amplifying nucleic acid molecules from an mRNA template(s) under conditions sufficient to increase the detection sensitivity and reliability through generation of secure and non-biaed cDNA molecules prior to gene-specific primer dependent amplification. Specifically, the invention relates to the use of oligo-dT or random primer or a mixture of oligo(dT) primer and random primer in a first-strand cDNA synthesis reaction. The invention also may comprises no random primer and oligo-dT depending on application purpose.

In one aspect of the invention, the buffer may comprise a monovalent cation selected from the group consisting of Na, K, NH₄, a magnesium salt, a reducing agent, nucleoside triphosphates, and at least one non-ionic detergent. The buffer may further comprise at least one primer suitable for priming reverse transcription of a template by the reverse transcriptase. The mixture may also comprise an RNase inhibitor protein. In one embodiment, the buffer comprises a potassium salt, a magnesium salt, nucleoside triphosphates, DTT, at least one or more reverse transcriptases, at least one or more DNA polymerases, at least one non-ionic detergent, BSA, and an RNase inhibitor protein.

In any of these methods and compositions, two or more reverse transcriptases and two or more DNA polymerases may be used, including any reverse transcriptase as described above.

Although the present invention is briefly summarized, the fuller understanding of the invention can be obtained by the following drawings, detailed description and appended claims. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

These and other features, aspects and advantages of the present invention will become better understood with reference to the accompanying drawings, wherein:

FIG. 1 shows freeze-thaw stability information of one-step RT-PCR mastermixes;

FIG. 2 shows lot-to-lot reproducibility of one-step RT-PCR mastermix;

FIG. 3 shows the detection sensitivity of one-step RT-PCR master mix using HeLa RNA with Gapdh-555 target; and

FIG. 4 shows performance audit of one-step RT-PCR mastermix.

### DETAILED DESCRIPTION OF THE INVENTION

The present invention relates to methods of increasing the efficiency of nucleic acid amplification, particularly, to increasing the sensitivity, accuracy, and reliability of gene expression analysis and quantitation. Thus, the present invention provides improved mRNA detection useful in gene discovery, genomic research, diagnostics and identification of differentially expressed genes and identification of genes of importance to disease. Other embodiments of the invention relate to stabilized and simplified easy-of-use antifreeze format mastermix solution improves the reliability of RT-PCR performance.

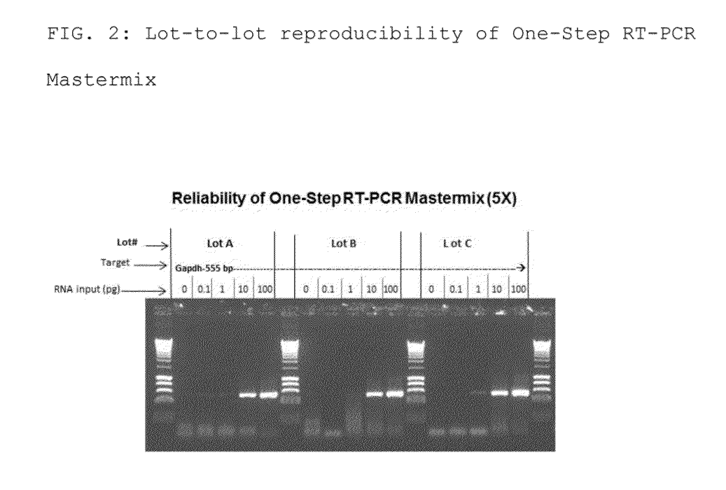

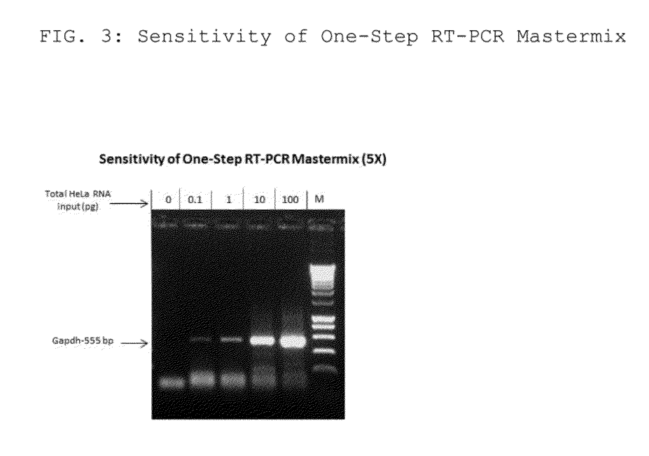

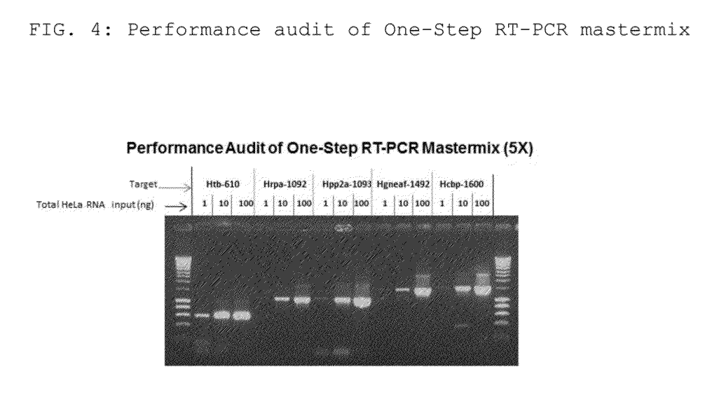

Other embodiments of the invention relate to stabilized concentrated reaction mixtures that simplify and improve the reliability of RT-PCR (FIG. 2, FIG. 3, and FIG. 4).

The reagent mixture of the present invention comprises a ready to use reagent solution that demonstrates prolonged stability when stored at −20 degree. The solution comprises (a) propylene glycol in a concentration between about 25% and about 45% by weight per volume; (b) a viral reverse transcriptase in a concentration sufficient for use in a reverse transcription reaction without adding additional reverse transcriptase wherein the viral reverse transcriptase, wherein the viral reverse transcriptase is selected from the group consisting of Avian Myeloblastosis Virus Reverse Transcriptase (AMV RT), Respiratory Syncytial Virus Reverse Transcriptase (RSV RT), Moloney Murine Leukemia Virus Reverse Transcriptase (MMLV RT), Human Immunodeficiency Virus Reverse Transcriptase (HIV RT), Equine Infectious Anemia Virus Reverse Transcriptase (EIAN RT), Rous-Associated Virus 2 Reverse Transcriptase (RAV2RT), Avian Sarcoma Leukosis Virus Reverse Transcriptase (ASLVRT), RNaseH (−) Reverse Transcriptase, SuperScript II Reverse Transcriptase, and ThermoScript Reverse Transcriptase; and (c)one or more DNA polymerases selected from the group consisting of Thermus Aquaticus (Taq), Thermus Thermophilus (Tth), Thermus Filiformis (Tfi), Thermus Brockianus (Tbr), Thermotoga Maritima (Tma), Thermotogo Neapolitana (Tne), Pyrococcus Furiosus (Pfu), Pyrococcus Woesei (Pwo), Vent, Deep Vent, Kodakaraensis, and mutants thereof, in a buffer suitable for use in a reverse transcription reaction which comprises a co-factor metal ion necessary for reverse transcriptase activity and nucleoside triphosphates.

The buffer may further comprise one or two primers suitable for priming reverse transcription of a template by the reverse transcriptase. The buffer may comprises an RNase inhibitor protein. The buffer may further comprise a bovine serum albumin. In addition, the buffer may comprise a potassium salt, a magnesium salt, nucleoside triphosphates, Dithiothreitol, at least one or two primers suitable for priming reverse transcription of a template by the reverse transcriptase, at least one non-ionic detergent, and an RNase inhibitor protein.

The solution may comprise one or two or more viral reverse transcriptase enzymes. The solution may comprise one or two or more DNA polymerase enzymes.

The buffer may comprise at least one random primer such as hexameric oligonucleotides, heptameric oligonucleotides, or octameric oligonucleotides, etc. The buffer may further comprise at least one gene-specific primer. In addition, the buffer may comprise at least one oligo deoxy-thymine primer such as deoxy-thymine-6-10 or deoxy-thymine-12-18. The buffer may comprise both random primer and oligo deoxy-thymine primer.

The metal ion necessary for reverse transcriptase activity may be magnesium ion (1-10 mM). In addition, the buffer may further comprise a monovalent cation and the monovalent cation may be K, Na, or NH₄ (50-500 mM).

The buffer may comprise a reducing agent and the reducing agent may be Dithiothreitol (1-10 mM). The buffer may further comprise a non-ionic detergent (e.g., NP-40, Tween-20, 0.01-1%). In addition, the buffer may comprise a trehalose in a concentration between about 3% and about 20% and/or a glycerol in a concentration between about 3% and about 20%. The buffer may further comprise a tracking dye (Xylene Cynol, Tartrazine, etc., optical density of 2 to 30).

The solution is stable for 12 months to 2 years when stored at about −20 degree. The solution shows better performances when the propylene glycol is in a concentration between about 30% and about 40%.

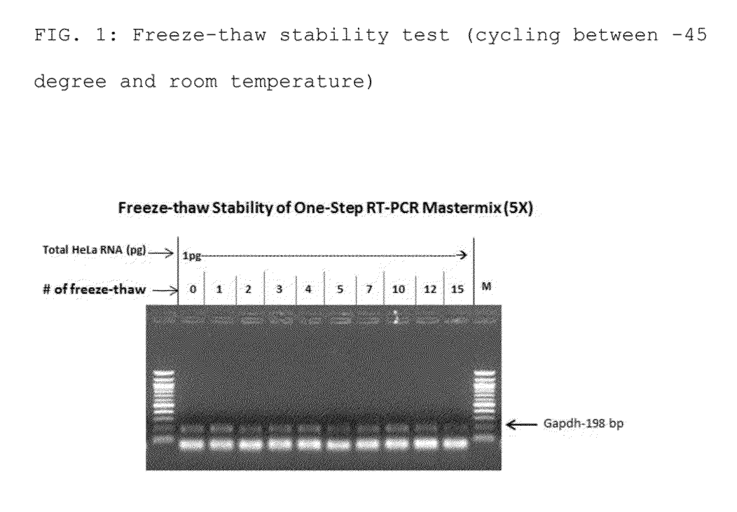

FIGS. 1 and 2 respectively show stability information of One-Step RT-PCR Mastermix and lot-to-lot reliability. Detection sensitivity as low as 0.1 μg total HeLa RNA with Gapdh-555 target is shown in FIG. 3. Robust detection capability of various mRNA targets with 1-100 ng total HeLa RNA is demonstrated as in FIG. 4. Propylene glycol based one-step RT-PCR mastermix is an antifreeze format formulation storage as low as −35 degree. It provides a highly sensitive, robust, easy to use, and convenient one-tube mastermix formulation. The 3× to 6× one-step RT-PCR mastermixes were found to be stable for more than 12 months when stored at −20° C.

Use of Primer Combinations in RT-PCR Mastermix

The present invention provide a more efficient, non-bias, and secure priming for cDNA synthesis prior to gene-specific PCR amplification. The use of optimal concentration and combinations of random primers (GSP) and oligo dT provides an efficient and representative conversion of mRNA sequences into cDNA in advance of GSP amplification. The length of oligo dT can vary from 6 bases to 25 bases and 6 bases to nine base random primers (e.g., hexameric, heptameric, octameric, etc.). The amount of random primers can vary from 10 ng to 200 ng for each reaction (25 μL) and oligo dT to be used can be 2 nM to 50 nM. The invention also comprises no random primer and no oligo-dT depending on application purpose. Pre-mix of blend of random hexamer and oligo dT one-step RT-PCR mastermix provides an enhanced detection sensitivity and cDNA yield as well as multiplex mRNA detection capacity.

Convenient, Stable, and Antifreeze Format Master Mix Compositions

Another embodiment is the form in which the reaction mixture is prepared and stably maintained. Traditionally, RT-PCR system components have been supplied as a number of separate components that are assembled into a complete reaction mix just prior to start of RT-PCR reaction primarily due to storage stability issue. A typical kit for RT-PCR contains the following components: Reaction Buffer (20-80 mM TrisCl pH 8.5, (or 50-100 mM Tris-SO4 pH 8.9), 30-100 mM KCl. d. 1-2 mM MgCl₂), 0.2 mM dNTPs, 2-20 units MMLV RT, 1-5 unit Taq DNA polymerase, and stabilizer.

Each of the above components is provided separately and frozen at −20 degree for storage. The general belief has been that the components cannot be mixed for long term storage. A key component of these systems is reverse transcriptase that is always stored in special storage buffer with at least 40-50% glycerol, and is only added to the reaction mix immediately prior to start of RT-PCR reaction. However, it is known that high percent of glycerol in one-step RT-PCR reduces detection sensitivity significantly, therefore, glycerol based one-step RT-PCR mastermix format was not yet successful. For this reason there is no report of successful formulation of complete mastermix of RT-PCR except thermostable Tth DNA polymerase based RT-PCR. However, unlike the case of viral reverse ranscriptases, Tth DNA polymerase is known to have very weak RT activity in the presence of Mn⁺⁺ and can be for limited application only.

For the first time, we have found that some or all of the components of the RT-PCR reaction can be combined and stored as a convenient ready-to-use mix that is stable to prolonged storage at −20 degree as an antifreeze format formulation. Propylene glycol based one-step RT-PCR mastermix will not freeze in storage as low as −35 degree and that can simply be added to a RNA template and GSP when assemble reactions. The ready to use reaction mixture may contain between about 25% and 45% propylene glycol to maintain stability of the RT enzyme(s) and DNA polymerases that is present in the mix.

Propylene glycol, also called propane-1,2-diol or propane-1,2-diol, is an organic compound with the chemical formula C₃H₈O₂. It is a viscous colorless, odorless and non-toxic liquid and able to lower the freezing point of water, and so it is used as antifreeze as well as aircraft de-icing fluid. It has been reported that the use of propylene glycol preserves viral RNA samples at room temperature for extended periods of time (Xianzhou Nie, et. al., Journal of Viological methods, Vol 175, pp 224-227, 2011). It is also claimed that adding up 20% propylene glycol will enhance sensitivity and specificity in PCR amplification. (Ping-Hug Teng, et. al., US2012/0244599, 2012) A combination study of small amounts of propylene glycol (1.5 mM less than 1%) and high concentrations of betaine (5.5M) has shown specificity improvement for high GC template DNA amplification (Fang Liu, et. al., Bioinformatics and Biomedical Engineering, 2009). However, there has been no prior publication reporting the use of propylene glycol as an essential stabilizing component in cDNA synthesis or RT-PCR amplification. We found that propylene glycol, unlike the case of glycerol, did not inhibit the RT activity even in the presence of high concentration propylene glycol and prevents from freezing of cDNA synthesis master even storage at −35 degrees.

Various format of RT-PCR mastermix can be successfully formulated (4× format to 10× format) for a variety of applications. The minimum components that may usefully be provided for the mixture are the propylene glycol, the RT, DNA polymerase, and a suitable buffer component. Suitable buffer compounds, such as Tris-HCl, Tris-SO₄, HEPES, Tricine etc, are well known in the art. Metal ions necessary for RT and DNA polymerase activity, such as Mg and a monovalent cation such as K, Na, and HN₄ may be present in concentrations that are suitable for RT and DNA polymerase activity upon addition to a template solution.

Additional components that may be present are a reducing agent, such as DTT, primer molecules such as gene specific primers (GSP), random primers of any suitable length, oligo(dT) compounds of any suitable length, anchored oligo(dT) molecules of suitable length, detergents or mixtures of detergents such as Tween, NP-40, Big Chap, Chapso. and equivalent reagents, dNTPs, and one or more RNase inhibitor proteins. The relative amounts contained in the mixture of such reagents necessary for use in RT-PCR reactions can be readily determined by the skilled artisan.

Accordingly, the present invention provides newly improved, convenient, and ready to use configurations for RT-PCR. The methods of the invention reduce the number of additions for assembly of RT-PCR reactions which is highly sought by researchers especially in high throughput applications.

According to the methods of the invention, the ready to use mixes for RT-PCR can be made at different concentrations and provided as 3× to 6× “mastermixes”. The following is an example of a 5× mastermix for RT-PCR that contains all components necessary for RT-PCR according to the methods of this invention. Using 5 uL of this masternix and RNA preparation of interest at a total volume of 25 uL provides a complete reaction mix for conversion and amplification of RNA molecules into cDNA and amplification of GSP target at the same time.

Formulation for 5× RT-PCR One-Step RT-PCR Mastermix:

5× One-Step RT-PCR Mastermix contains random hexamer and oligo dT and tracking Dye: 300 mM Tris-HCl, pH 8.5, 150 mM KCl), 8 mM MgCl₂ 1.0 mM dNTP (each), 25 nM oligo(dT)₂₀, 20 ng random hexamer, 40% Propylene glycol, 0.5% Chapso, 6 units of MMLV(RnaseH-) RT, 2 units of Taq DNA polymerase, and blue dye (15 OD615).

In addition to the above formulation, three other mastermixes were prepared that contained all reagents except the primers.
RT-PCR Mastermix 1 did not have random primers.
RT-PCR Mastermix 2 did not have oligo dT primers.
RT-PCR Mastermix 3 did not have both random primer and oligo-dT.

All of the above 5× RT-PCR mastermixes were found to be stable at least 12 months when stored at −20° C. FIG. 4 shows the results and the efficacy of RT-PCR products with these mastermixes.

It will be evident to those skilled in the art that a variety of different reverse transcriptases can be used according to the method of the invention. The reverse transcriptases may include, without limitation, AMV RT, RSV RT, MMLV RT, RNase H-mutants of various reverse transcriptases, HIV RT, EIAV RT, RAV2 RT, TTH DNA polymerase, C.hydrogenoformans DNA polymerase, Superscript II RT, SuperScript RT, ThermoScript RT and mixtures thereof. It will also be obvious that one or more of the components of the above mastermix can be substituted with other equivalent reagent or protein. For example, there are a number of different RNase inhibitor proteins that can be used. Thermostable DNA polymerases suitable for use in the mastermixes are well known in the art and include Taq, Tth, Tne, Tma, Tli, Pfu, Pwo, Bst, Bca, Sac, Tac, Tfl, Tru, Mth, Mtb, and Mlep DNA polymerases and the like.

The composition of the 5× buffer provided can also be varied, for example, by use of other buffers such as sulfate containing buffers or acetate based buffers that have been used for RT-PCR. It will be apparent to those skilled in the art that different formulations can be optimized for different applications.

A variety of formulations have been described for One-step RT PCR, however, in all cases the buffers and enzymes are kept separately and are only mixed immediately prior to reverse transcription reaction. According to the methods of the invention, the reverse transcriptase, Tag DNA polymerase and buffers, dNTP's, co-factors and all other components for one step RT PCR can be mixed together in a variety of different concentrations to provide a ready to use antifreeze format mastermix formulation.

### EXAMPLES

FIG. 1: Freeze-thaw Stability Test

Test samples were 1-Step RT-PCR Mastermix (5×). Samples were subjected to number of freeze-thaw cycles as indicated. This 5× mastermix is an antifreeze format formulation storage as low as −35 degree so used ethanol-dry ice bath (−78 degrees) to test freeze thaw stability. Reactions were assembled on ice by adding 1 μg of total HeLa RNA, and GAPDH-198bp GSP primers and 5× One-Step RT-PCR Mastermix and carried out RT-PCR amplification reaction (incubation at 45 degree for 30 minutes and followed by 40 cycles of 94 degree, 15 s, 60 degree, 30 s, 68 degree, 1 min) and analyzed amplified product by 1% agarose gel electrophoresis. RT-PCR amplification was carried out on the thermocycler in the following continuous order: 45° C. for 30 minutes then 40 cycles of PCR amplification (94° C. for 15 s, 60° C. for 30 s, 68° C. for 1 min). The amplified RT-PCR product was analyzed by 1% agarose gel electrophoresis.

FIG. 2: Lot-to-lot Reliability of One-Step RT-PCR Mastermix

Test of 3 different lot samples were: (1). Lot A, (2). Lot B, and (3). Lot C one-step RT-PCR mastermix (5× formulation). Reactions were assembled on ice by adding 0.1 to 100 μg of total HeLa RNA as indicated, Gapdh-555 primers and 5× One-Step RT-PCR Mastermixes (25 ul volume). RT-PCR amplification was carried out on the thermocycler in the following continuous order: 45° C. for 30 minutes then 40 cycles of PCR amplification (94° C. for 15 s, 60° C. for 30 s, 68° C. for 1 min). The amplified RT-PCR product was analyzed by 1% agarose gel electrophoresis.

FIG. 3: Sensitivity of One-Step RT-PCR Mastermix

Reactions were assembled on ice by adding 0.1 to 100 pg of total HeLa RNA as indicated and Gapdh-555 primers and 5× One-Step RT-PCR Mastermix contains random hexamer, oligo dT and tracking dye (25 ul volume). RT-PCR amplification was carried out on the thermocycler in the following continuous order: 25° C. for 5 minutes, 45° C. for 30 minutes then 40 cycles of PCR amplification (94° C. for 15 s, 60° C. for 30 s, 68° C. for 1 min). The amplified RT-PCR product was analyzed by 1% agarose gel electrophoresis.

FIG. 4: Performance of One-Step RT-PCR Mastermix

Reactions were assembled on ice by adding 1-100 ng of total HeLa RNA as indicated and Htb-610, Hrpa-1092, Hpp2a-1093, Hgneaf-1494, Hcbp-1600 primers and 5× One-Step RT-PCR Mastermix contains random hexamer, oligo dT and tracking dye (25 ul volume). RT-PCR amplification was carried out on the thermocycler in the following continuous order: 25° C. for 5 minutes, 45° C. for 30 minutes then 40 cycles of PCR amplification (94° C. for 15 s, 60° C. for 30 s, 68° C. for 1.5 min). The amplified RT-PCR product was analyzed by 1% agarose gel electrophoresis.

While the invention has been shown and described with reference to different embodiments thereof, it will be appreciated by those skilled in the art that variations in form, detail, compositions and operation may be made without departing from the spirit and scope of the invention as defined by the accompanying claims.

What is claimed is:

1. A reagent mixture comprising a ready to use reagent solution, wherein the solution comprises:
(a) propylene glycol in a concentration between about 25% and about 45%;
(b) a viral reverse transcriptase in a concentration sufficient for use in a reverse transcription reaction without adding additional reverse transcriptase wherein the viral reverse transcriptase is selected from the group consisting of Avian Myeloblastosis Virus Reverse Transcriptase, Respiratory Syncytial Virus Reverse Transcriptase, Moloney Murine Leukemia Virus Reverse Transcriptase, Human Immunodeficiency Virus Reverse Transcriptase, Equine Infectious Anemia Virus Reverse Transcriptase, Rous-Associated Virus 2 Reverse Transcriptase, Avian Sarcoma Leukosis Virus Reverse Transcriptase, RNaseH (−) Reverse Transcriptase, SuperScript II Reverse Transcriptase, and ThermoScript Reverse Transcriptase; and
(c) one or more DNA polymerases selected from the group consisting of Thermus Aquaticus, Thermus Thermophilus, Thermus Filiformis, Thermus Brockianus, Thermotoga Maritima, Thermotogo Neapolitana, Pyrococcus Furiosus, Pyrococcus Woesei, Vent, Deep Vent, Kodakaraensis, and mutants thereof, in a buffer suitable for use in a reverse transcription reaction, wherein the buffer comprises:
a co-factor metal ion necessary for reverse transcriptase activity; and
nucleoside triphosphates.

2. The mixture according to claim 1, wherein the buffer further comprises at least one primer suitable for priming reverse transcription of a template by the reverse transcriptase.

3. The mixture according to claim 1, wherein the buffer further comprises an RNase inhibitor protein.

4. The mixture according to claim 1, wherein the buffer further comprises bovine serum albumin.

5. The mixture according to claim 1, wherein the buffer further comprises a potassium salt, a magnesium salt, nucleoside triphosphates, Dithiothreitol, at least one or two primers suitable for priming reverse transcription of a template by the reverse transcriptase, at least one non-ionic detergent, and an RNase inhibitor protein.

6. The mixture according to claim 1, wherein the solution further comprises at least one viral reverse transcriptase enzyme.

7. The mixture according to claim 1, wherein the solution further comprises at least one DNA polymerase enzyme.

8. The mixture according to claim 1, wherein the buffer further comprises at least one random primer and the random primer is hexameric oligonucleotides, heptameric oligonucleotides, or octameric oligonucleotides.

9. The mixture according to claim 1, wherein the buffer further comprises at least one gene-specific primer.

10. The mixture according to claim 1, wherein the buffer further comprises at least one oligo deoxy-thymine primer and the oligo deoxy-thymine primer is deoxy-thymine-6-10 or deoxy-thymine-12-18.

11. The mixture according to claim 1, wherein the buffer further comprises at least one random primer and at least one oligo deoxy-thymine primer.

12. The mixture according to claim 1, wherein the co-factor metal ion is magnesium ion.

13. The mixture according to claim 1, wherein the buffer further comprises a monovalent cation and the monovalent cation is K, Na, or NH₄.

14. The mixture according to claim 1, wherein the buffer further comprises a reducing agent.

15. The mixture according to claim 1, wherein the buffer further comprises a non-ionic detergent and the non-ionic detergent is NP-40, Tween-20, Big Chap, or Chapso.

16. The mixture according to claim 1, wherein the buffer further comprises a trehalose in a concentration between about 3% and about 20%.

17. The mixture according to claim 1, wherein the buffer further comprises a glycerol in a concentration between about 3% and about 20%.

18. The mixture according to claim 1, wherein the buffer further comprises a tracking dye.

19. The mixture according to claim 1, wherein the solution is stable for 12 months to 2 years when stored at about −20° C.

20. The mixture according to claim 1, wherein the propylene glycol is in a concentration between about 30% and about 40%.
